SEQUENCE LISTING (9 sequences)

SEQ ID NO: 1 - protein, 101 aa, Homo sapiens
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 2 - protein, 501 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 3 - protein, 46 aa, Artificial Sequence [sequence omitted]

SEQ ID NO: 4 - protein, 478 aa, Artificial Sequence
  BACE1 N-terminus
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVC

SEQ ID NO: 5 - DNA, 19 bp, Artificial Sequence
  SUMO1 siRNA #2
aacacatctcaagaaactc

SEQ ID NO: 6 - DNA, 28 bp, Artificial Sequence
  BACE1 C-terminus forward primer
taagggcccaaccctcatgaccatagcc

SEQ ID NO: 7 - DNA, 20 bp, Artificial Sequence
  BACE1 C-terminus reverse primer
tggcaagtgtagcggtcacg

SEQ ID NO: 8 - DNA, 21 bp, Artificial Sequence
  BACE1 N-terminus forward primer
cgtcaatgacggtaaatggcc

SEQ ID NO: 9 - DNA, 28 bp, Artificial Sequence
  BACE1 N-terminus reverse primet
tactcgagcacaccatgaggcagagtgg

(19) United States
(12) Patent Application Publication
(10) Pub. No.: US 2014/0186357 A1
(43) Pub. Date: Jul. 3, 2014

(54) METHOD FOR TREATMENT OF DEGENERATIVE BRAIN DISORDERS COMPRISING INHIBITOR OF SUM01 AND BACE1 INTERACTION AS AN ACTIVE INGREDIENT

(71) Applicant: KOREA CENTER FOR DISEASE CONTROL AND PREVENTION, CHUNGCHEONGBUK-DO (KR)

(72) Inventors: Young Ho Koh, Daejeon (KR); Sang Moon Yun, Chungcheongbuk-do (KR); Sun-Jung Cho, Chungcheongbuk-do (KR); Chulman Jo, Chungcheongbuk-do (KR); Sangmee Ahn, Chungcheongnam-do (KR); Jae Chun Song, Chungcheongbuk-do (KR); Sung Yeon Song, Chungcheongbuk-do (KR)

(73) Assignee: KOREA CENTER FOR DISEASE CONTROL AND PREVENTION, CHUNGCHEONGBUK-DO (KR)

(21) Appl. No.: 14/021,210

(22) Filed: Sep. 9, 2013

(30) Foreign Application Priority Data
Dec. 31, 2012 (KR) 10-2012-0158455

(51) Int. Cl. C12N 15/113; G01N 33/50; G01N 33/68; C12Q 1/68; A61K 38/02; A61K 39/395

(52) U.S. Cl. CPC C12N 15/1138; A61K 38/02; A61K 39/3955; G01N 33/6896; C12Q 1/68; G01N 33/5044
USPC 424/139.1; 514/17.7; 514/17.8; 514/44 A; 435/7.92; 436/501; 506/9; 435/6.12

(57) ABSTRACT

A treatment method for degenerative brain disorders using a pharmaceutically effective dose of the inhibitor of SUMO1 (small ubiquitin-like modifier 1) and BACE1 (β-secretase) interaction, or the inhibitor of SUMO1 expression or activation is provided. More specifically, it was confirmed that SUMO1 increased BACE1 accumulation and Aβ generation, that is SUMO1 regulated BACE1 accumulation by interacting with BACE1, and BACE1 dileucine motif was involved in SUMO1-mediated BACE1 accumulation. In addition, SUMO1 protein induced autophagy in H4 cells, while SUMO1 depletion reduced LC3-II level. It was further confirmed that SUMO1 and LC3 were co-localized in the cortex of APP transgenic mice. As shown herein, a pharmaceutically effective dose of the inhibitor of SUMO1 and BACE1 interaction or the inhibitor of SUMO1 expression can be effectively used for the treatment of degenerative brain disorders.

### CROSS-REFERENCES TO RELATED APPLICATION

This patent application claims the benefit of priority from Korean Patent Application No. 10-2012-0158455, filed on Dec. 31, 2012, the contents of which are incorporated herein by reference.

### BACKGROUND OF THE INVENTION

1. Field of the Invention

The present invention relates to a treatment method for degenerative brain disorders using a pharmaceutically effective dose of the inhibitor of SUMO1 (small ubiquitin-like modifier 1) and BACE1 (β-secretase) interaction or the inhibitor of SUMO1 expression or activation.

2. Description of the Related Art

One of the common observations of neurodegenerative diseases, including Alzheimer's disease (AD) and Huntington's disease, is an accumulation of proteins that is considered to be involved in pathological processes. Several proteins such as parkin, ubiquitin carboxy-terminal hydrolase (UCH-L1) or small ubiquitin-like modifier (SUMO) have been observed in neurodegenerative diseases (Kitada et al., 1998; Leroy et al., 1998; Ueda et al., 2002).

While ubiquitination targets proteins for degradation, sumoylation modifies the interaction of target proteins with protein partners and their activity, stability, and subcellular localization (Gill, 2003; Pichler and Melchior, 2002). SUMO paralogs (SUMO1, SUMO2 and SUMO3) had a wide expression in all tissues and can be covalently linked to target proteins to alter their cellular distribution, function, and metabolism (Schwartz and Hochstrasser, 2003). SUMO1 is an 11-kDa protein that is 18% identical to ubiquitin (Muller et al., 2001), SUMO2 and SUMO3 differ only in 3 N-terminal residues (Wilkinson et al., 2010). SUMO modification plays an important role in protein trafficking, nuclear bodies, the ubiquitin-proteasome system, and apoptosis. Many studies have suggested that some proteins may be preferentially sumoylated by specific SUMO types (Gregoire and Yang, 2005; Vertegaal et al., 2006).

Many SUMO target proteins have been identified in neurons that are cytosolic or membrane proteins, including glutamate receptor subunit 6 (Martin et al., 2007). Although SUMO can be covalently linked to specific proteins, it has been reported that SUMO even in the absence of the C-terminal diglycine motif can participate in protein-protein interaction (Yan et al., 2010).

Both extracellular β-amyloid (Aβ) deposits and the origin of β-secretase (BACE1) accumulation and its temporal relationship with neuritic pathology remain issues of ongoing debate in neurodegenerative disease research. Several molecules implicated in neurodegenerative diseases including AD have been identified as SUMO targets. Tau and amyloid precursor protein (APP) have been shown to be modified by SUMO (Dorval and Fraser, 2006; Zhang and Sarge, 2008). Strong SUMO2/3 immunoreactivity has been reported in AD neurons (Martin et al., 2007), and SUMO-positive deposits were detected in APP transgenic mice (Tg 2576) (Takahashi et al., 2008). Recently, the present inventors reported that Ubc9 polymorphisms were associated with late onset AD patients in the Korean population (Ahn et al., 2009). Two previous studies indicated that SUMO3 over-expression affects Aβ levels (Dorval et al., 2007; Li et al., 2003). However, the meaning of their results is not clear as the 2 studies observed opposite effects of SUMO over-expression on Aβ levels. Thus far, however, the role of SUMO in AD is still somewhat controversial.

BACE1 is a type I integral membrane-associated aspartyl protease (Vassar et al., 1999). An increase in BACE1 activity has been shown to be correlated with brain Aβ production in the frontal cortex (Li et al., 2004). Previously, the present inventors reported that BACE1 was degraded via the lysosomal pathway and that the dileucine motif was important to regulate BACE1 levels (Koh et al., 2005). Notably, the dileucine motif in BACE1 interacts with the Golgi-localized, γ-ear-containing adenosine diphosphate riboxylation factor-binding (GGA) family. Although depletion of GGA3 during apoptosis led to an increase in BACE1 levels (Tesco et al., 2007), previous studies showing BACE1 elevation in the presence or absence of cell death imply that BACE1 accumulation was correlated with amyloid pathology (Zhao et al., 2007).

Autophagy is a main mechanism for maintaining cellular homeostasis. It is mediated via the degradation and recycling of cellular proteins and aids cell survival on exposure to internal or external cellular stresses. The autophagic process starts with the entrapment of material by a double-membrane vesicle called the autophagosome. Autophagosomes are selectively associated with LC3-II, an isoform of microtubule-associated protein LC3. Upon the induction of autophagy, LC3-I is conjugated with phosphatidylethanolamine (PE) to generate LC3-II. Recently, autophagy has been implicated in a number of diseases, including neurodegenerative diseases, Alzheimer's disease (AD), Parkinson's disease (PD), lysosomal storage diseases, and cancer.

The four drugs officially approved as treating agents for Alzheimer's disease by Food and Drug Administration (FDA), USA, which are Tacrine, Rivastigmine, Donepezil, and Galantamine, are all acetylcholinesterase inhibitors that are functioning to improve cognitive function by inhibiting the activity of acetylcholinesterase. However, these drugs only demonstrate temporary alleviation of symptoms in some of Alzheimer's disease patients (40˜50%) and the medicinal effect of them does not last long. degenerative brain disorders characteristically requires a long-term treatment, but those acetylcholinesterase inhibitors developed so far for the disease have been proved to have a problem of carrying various side effects including liver toxicity over the long-term administration. The biggest barrier in the development of Alzheimer's disease treating agents is uncertainty of cause of the disease. According to the recent advancement of genetics, cell biology, and molecular biology, amyloid hypothesis has been proposed as the onset mechanism of Alzheimer's disease. It has also been proved that Aβ accumulation in the brain and the following neurotoxicity are major causes of Alzheimer's disease. Therefore, studies have been focused on the screening of materials that have the inhibitory effect on Aβ generation and coagulation and the activity of suppressing toxicity with less side effects, like BACE1 inhibitor, etc.

The present inventors have tried to develop a composition for the prevention and treatment of neurodegenerative diseases, and in the course identified SUMO1 to be a BACE1-interaction partner and confirmed that SUMO1 increased BACE1 accumulation and Aβ generation and regulated the level of BACE1 by interacting with BACE1. The inventors also confirmed that the dileucine motif of BACE1 was involved in BACE1 accumulation. In addition, the present inventors confirmed that SUMO1 induced autophagic activation in H4 cells and the depletion of SUMO1 decreased the level of LC3-II. The present inventors also observed that SUMO1 was co-localized with LC3 in APP transgenic mice. As a result, the present inventors completed this invention by confirming that a pharmaceutically effective dose of the inhibitor of SUMO1 and BACE1 interaction or the inhibitor of SUMO1 expression could be effectively used for the treatment of neurodegenerative disease.

### SUMMARY OF THE INVENTION

It is an object of the present invention to provide a treatment method for degenerative brain disorders containing the step of administering a pharmaceutically effective dose of the inhibitor of SUMO1 (small ubiquitin-like modifier 1) and BACE1 (β-secretase) interaction or the inhibitor of SUMO1 expression or activation to a subject having degenerative brain disorders.

It is another object of the present invention to provide a diagnostic method for degenerative brain disorders containing the step of measuring the levels of SUMO1 and BACE1 interaction or SUMO1 expression.

In addition, it is also an object of the present invention to provide a screening method for candidates for treating degenerative brain disorders containing the step of measuring the levels of SUMO1 and BACE1 interaction or SUMO1 expression.

To achieve the above objects, the present invention provides a treatment method for degenerative brain disorders containing the step of administering a pharmaceutically effective dose of the inhibitor of SUMO1 (small ubiquitin-like modifier 1) and BACE1 (β-secretase) interaction to a subject having degenerative brain disorders.

The present invention also provides a treatment method for degenerative brain disorders containing the step of administering a pharmaceutically effective dose of the inhibitor of SUMO1 expression or activation to a subject having degenerative brain disorders.

The present invention further provides a pharmaceutical composition for the prevention and treatment of degenerative brain disorders comprising the inhibitor of SUMO1 and BACE1 interaction as an active ingredient.

The present invention also provides a pharmaceutical composition for the prevention and treatment of degenerative brain disorders comprising the inhibitor of SUMO1 expression or activation as an active ingredient.

The present invention provides a diagnostic method for degenerative brain disorders comprising the following steps:

1) measuring the level of SUMO1 and BACE1 interaction in a sample separated from a test subject;

2) selecting a subject demonstrating the increased level of SUMO1 and BACE1 interaction, measured in step 1), compared with that of the normal control; and

3) evaluating the risk of degenerative brain disorders in the selected subject of step 2).

The present invention also provides a diagnostic method for degenerative brain disorders comprising the following steps:

1) measuring the level of SUMO1 expression in a sample separated from a test subject;

2) selecting a subject demonstrating the increased level of SUMO1 expression, measured in step 1), compared with that of the normal control; and

3) evaluating the risk of degenerative brain disorders in the selected subject of step 2).

The present invention also provides a screening method for candidates for treating degenerative brain disorders comprising the following steps:

1) treating a test material to cells expressing SUMO1 and BACE1;

2) measuring the level of SUMO1 and BACE1 interaction in the cells of step 1); and

3) selecting a test material that was able to reduce the level of SUMO1 and BACE1 interaction, measured in step 2), compared with the non-treated control.

The present invention also provides a screening method for candidates for treating degenerative brain disorders comprising the following steps:

1) treating a test material to cells expressing SUMO1 and BACE1;

2) measuring the level of SUMO1 expression in the cells of step 1); and

3) selecting a test material that was able to reduce the level of SUMO1 expression, measured in step 2), compared with the non-treated control.

The present invention also provides a screening method for candidates for treating degenerative brain disorders comprising the following steps:

1) treating a test material to the cells obtained from a test subject;

2) measuring the level of SUMO1 and BACE1 interaction in the cells of step 1); and

3) selecting a test material that was able to reduce the level of SUMO1 and BACE1 interaction, measured in step 2), compared with the non-treated control.

The present invention also provides a screening method for candidates for treating degenerative brain disease comprising the following steps:

1) treating a test material to the cells obtained from a test subject;

2) measuring the level of SUMO1 expression in the cells of step 1); and 3) selecting a test material that was able to reduce the level of SUMO1 expression, measured in step 2), compared with the non-treated control.

The present invention also provides a screening method for candidates for treating degenerative brain disorders comprising the following steps:

1) treating a test material to SUMO1 and BACE1 proteins;

2) measuring the level of SUMO1 and BACE1 interaction in the cells of step 1); and

3) selecting a test material that was able to reduce the level of SUMO1 and BACE1 interaction, measured in step 2), compared with the non-treated control.

The present invention also provides a screening method for candidates for treating degenerative brain disorders comprising the following steps:

1) treating a test material to SUMO1 and BACE1 proteins;

2) measuring the level of SUMO1 expression in the cells of step 1); and

3) selecting a test material that was able to reduce the level of SUMO1 expression, measured in step 2), compared with the non-treated control.

### Advantageous Effect

As explained hereinbefore, the inhibitor of SUMO1 (small ubiquitin-like modifier 1) and BACE1 (β-secretase) interaction or the inhibitor of SUMO1 expression of the present invention significantly inhibits the generation of β-amyloid (Aβ), one of the major causes of degenerative brain disorders, hence it can be effectively used as a pharmaceutical composition for the prevention and treatment of degenerative brain disorders.

### BRIEF DESCRIPTION OF THE DRAWINGS

The application of the preferred embodiments of the present invention is best understood with reference to the accompanying drawings, wherein:

FIG. 1A and FIG. 1B are diagrams illustrating the levels of SUMO1 in the amyloid precursor protein (APP) transgenic mice.

WT: negative control, and

Tg: APP transgenic mouse.

FIG. 1C is a diagram illustrating the correlation of amyloid plaque and SUMO expression site.

WT: negative control, and

Tg: APP transgenic mouse.

FIG. 2A and FIG. 2B are diagrams illustrating the inducement of SUMO1 expression by Aβ₁₋₄₀ peptides.

FIG. 2C and FIG. 2D are diagrams illustrating the SUMO1-conjugated protein expression induced by Aβ₁₋₄₀ peptides.

FIG. 3A is a diagram illustrating the up-regulation of BACE1 by SUMO1.

FIG. 3B and FIG. 3C are diagrams illustrating the up-regulation of BACE1 by wild-type SUMO1 or mutant SUMO1.

FIG. 3D is a diagram illustrating the interaction between SUMO1 and BACE1.

FIG. 3E is a diagram illustrating the BACE1 site specifically binding to SUMO1, confirmed by using wile-type BACE1 and mutant BACE1.

FIG. 3F is a diagram illustrating the BACE1 expression induced by SUMO1 in the cells expressing mutant BACE1.

FIG. 4A and FIG. 4B are diagrams illustrating the levels of BACE1 changed by SUMO1 depletion in the cells expressing wild-type BACE1 and mutant BACE1.

FIG. 4C is a diagram illustrating the inhibition of BACE1 up-regulation induced by Aβ₁₋₄₀ peptides under the condition of SUMO1 depletion.

FIG. 4D and FIG. 4E are diagrams illustrating the role of SUMO1 in the STS-mediated BACE1 accumulation.

FIG. 4F is a diagram illustrating the accumulation of BACE1 induced by STS-mediated apoptosis in the SUMO1 knockdown mice.

FIG. 5A and FIG. 5B are diagrams illustrating the expressions of Aβ₁₋₄₀ peptides induced by SUMO1 in the cells expressing wild-type BACE1 and mutant BACE1.

FIG. 5C and FIG. 5D are diagrams illustrating the expressions of Aβ₁₋₄₀ peptides induced by SUMO1 in the cells expressing wild-type BACE1 and mutant BACE1.

FIG. 6A is a diagram illustrating the C-terminal deleted BACE1-NTF and N-terminal deleted BACE1-CTF.

BACE1 wt: wild-type BACE1,

BACE1-CTF: BACE1 C-terminal fragment, residues 456-501, and

BACE1-NTF: BACE1 N-terminal fragment, residues 1-478.

FIG. 6B is a diagram illustrating the generation of Aβ₁₋₄₀ peptides by BACE1-NTF and BACE1-CTF constructed in the above.

BACE1 wt: wild-type BACE1,

BACE1-CTF: BACE1 C-terminal fragment, residues 456-501, and

BACE1-NTF: BACE1 N-terminal fragment, residues 1-478.

FIG. 7 is a diagram illustrating the on-set of Alzheimer's disease by the positive feedback loop among SUMO1, BACE1, and Aβ.

FIG. 8 is a diagram illustrating the up-regulation of LC3-II in the presence of SUMO1.

FIG. 9 is a diagram illustrating the increase of autophagosome formation in the presence of SUMO1.

FIG. 10 is a diagram illustrating the plenty of autophagosomes in SUMO1-transfected H4 cells (HGS1)

FIG. 11 is a diagram illustrating the down-regulation of LC3-II by SUMO1 depletion.

H4 shC: positive control, and

H4 shS1: SUMO1 shRNA-treated group.

FIG. 12A is a diagram illustrating the down-regulation of Aβ by the treatment of 3-methyladenine.

CTR: control, and

3MA: 3-methyladenine-treated group.

FIG. 12B is a diagram illustrating the down-regulation of LC3 by the treatment of 3-methyladenine.

Control: control, 3-MA: 3-methyladenine-treated group,

FS: incubated in full-serum, and

SD: incubated in serum-depleted.

FIG. 12C is a diagram illustrating the recovery of decreased Aβ expression mediated by 3-methyladenine in the presence of SUMO1.

CTR: control, and

3-MA: 3-methyladenine-treated group.

FIG. 13A is a diagram illustrating the up-regulation of LC3-II in the APP transgenic mice.

WT1: wild-type 1,

Tg1: transgenic mouse 1,

WT2: wild-type 2,

Tg2: transgenic mouse 2,

WT3: wild-type 3, and

Tg3: transgenic mouse 3.

FIG. 13B is a diagram illustrating the up-regulation of LC3-II in the APP transgenic mice.

WT: wild-type, and

Tg: transgenic mouse.

FIG. 13C is a diagram illustrating the co-localization of LC3 and SUMO1 in the cortex of APP transgenic mice.

WT: wild-type, and

Tg: transgenic mouse.

### DESCRIPTION OF THE PREFERRED EMBODIMENTS

Hereinafter, the present invention is described in detail.

The present invention provides a treatment method for degenerative brain disorders containing the step of administering a pharmaceutically effective dose of the inhibitor of SUMO1 (small ubiquitin-like modifier 1) and BACE1 (β-secretase) interaction to a subject having degenerative brain disease.

The present invention also provides a treatment method for degenerative brain disorders containing the step of administering a pharmaceutically effective dose of the inhibitor of SUMO1 expression or activation to a subject having degenerative brain disease.

The present invention further provides a pharmaceutical composition for the prevention and treatment of degenerative brain disorders comprising the inhibitor of SUMO1 and BACE1 interaction as an active ingredient.

The present invention also provides a pharmaceutical composition for the prevention and treatment of degenerative brain disorders comprising the inhibitor of SUMO1 expression or activation as an active ingredient.

The said SUMO1 and BACE1 are human originated proteins and preferably obtained from the same species, but not always limited thereto.

The SUMO1 herein is preferably composed of the amino acid sequence represented by SEQ. ID. NO: 1, but not always limited thereto.

The full length BACE1 is composed of the amino acid sequence represented by SEQ. ID. NO: 2, or the C-terminal deleted sequence from the original BACE1 sequence represented by SEQ. ID. NO: 4, but not always limited thereto.

The said inhibitor of SUMO1 and BACE interaction is preferably selected from the group consisting of peptides binding complementarily to SUMO1 or BACE1, peptides binding complementarily to the fragment having C-terminal deleted from the BACE1 sequence represented by SEQ. ID. NO: 3, peptide mimetics, substrate analogs, aptamers and antibodies, but not always limited thereto.

The said inhibitor of SUMO1 expression or activation is preferably selected from the group consisting of antisense nucleotides binding complementarily to SUMO1 mRNA, siRNA, and shRNA, but not always limited thereto.

The said degenerative brain disorders is preferably the one induced by the accumulation of Aβ (β-amyloid), which is preferably the one selected from the group consisting of dementia, Alzheimer's disease, stroke, Huntington's disease, Pick's disease, and Creutzfeldt-Jakob disease, and more preferably the one selected from the group consisting of dementia, Alzheimer's disease, and Huntington's disease.

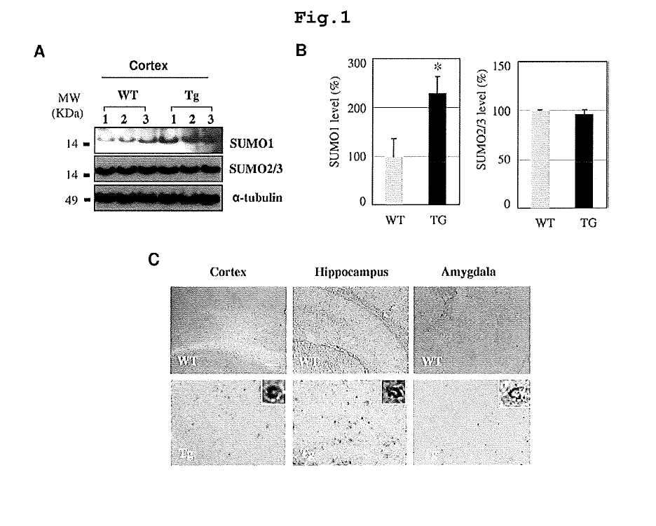

In a preferred embodiment of the present invention, the levels of SUMO1 in the transgenic mice with Alzheimer's disease were measured. As a result, the levels of SUMO1 were significantly increased in the experimental group having Alzheimer's disease, compared with the normal control group. The correlation between amyloid plaque and SUMO1 expression site was also investigated. As a result, it was confirmed that SUMO1 and amyloid plaque were co-localized in the same region (see FIG. 1).

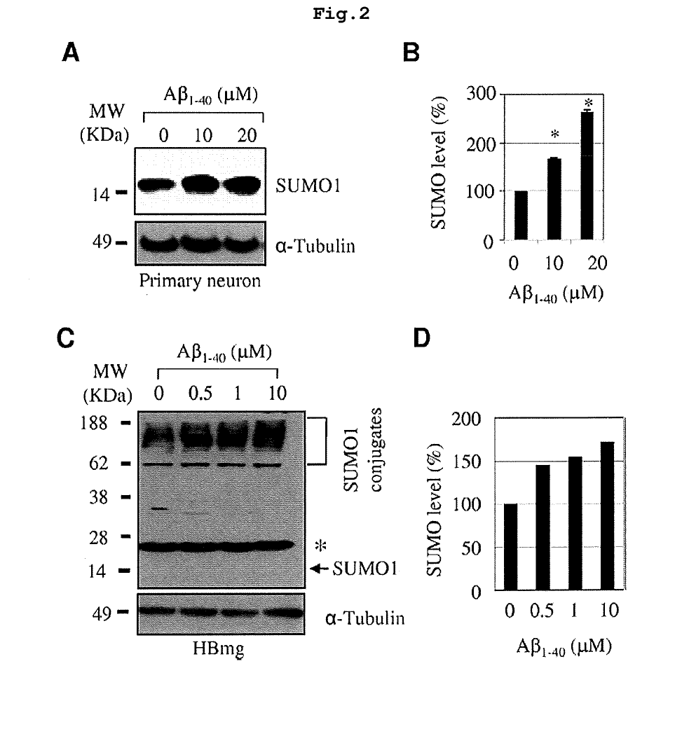

The present inventors also investigated the effect of Aβ₁₋₄₀ peptides on the levels of SUMO1 and BACE1. As a result, the levels of SUMO1 and SUMO1-conjugated proteins were significantly increased by Aβ₁₋₄₀ peptides (see FIG. 2).

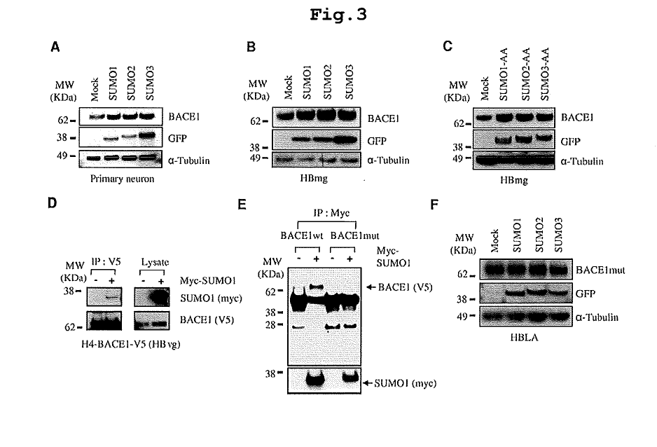

The present inventors also investigated the effect of SUMO1 on the levels of BACE1. As a result, it was confirmed that the levels of BACE1 were increased by SUMO1, which was attributed to the interaction of SUMO1 with the dileucine motif of BACE1 (see FIG. 3).

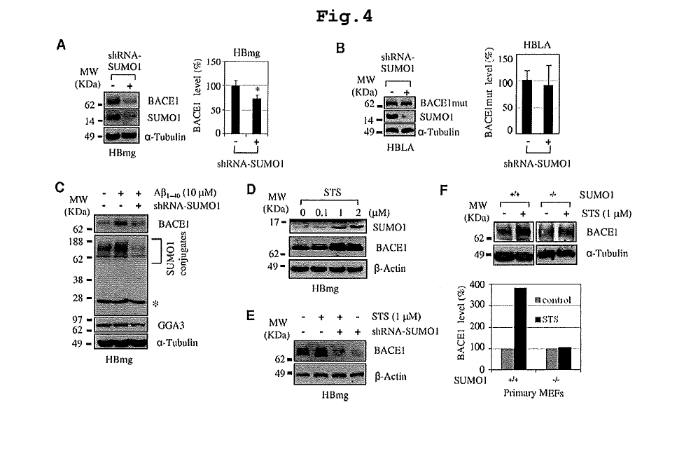

The present inventors further investigated the effect of SUMO1 depletion on the levels of BACE1. As a result, it was confirmed that the levels of BACE1 were significantly reduced by the depletion of SUMO1 (see FIG. 4).

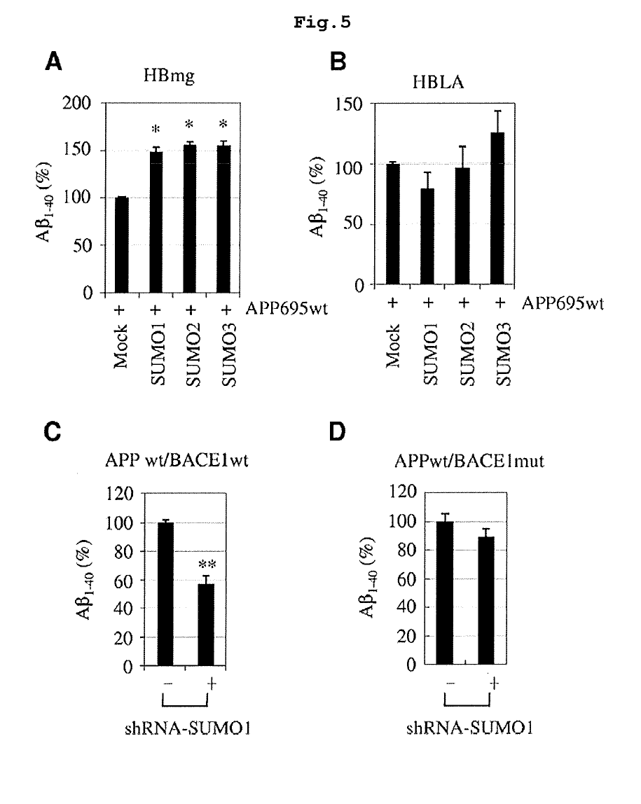

The present inventors also investigated whether SUMO1 could increase the generation of Aβ. As a result, it was confirmed that SUMO1 promoted the generation of Aβ, but did not affect the generation of Aβ in the cells expressing mutant BACE1 having modified dileucine motif. Therefore, it was confirmed that the SUMO1-mediated Aβ generation was dependant to BACE1 expression, which was regulated by the dileucine motif of BACE1 (see FIG. 5A and FIG. 58).

The present inventors also investigated the effect of SUMO1 depletion on the levels of Aβ. As a result, it was confirmed that the levels of Aβ were significantly decreased by the depletion of SUMO1 in the cells expressing BACE1 (see FIG. 5C and FIG. 5D).

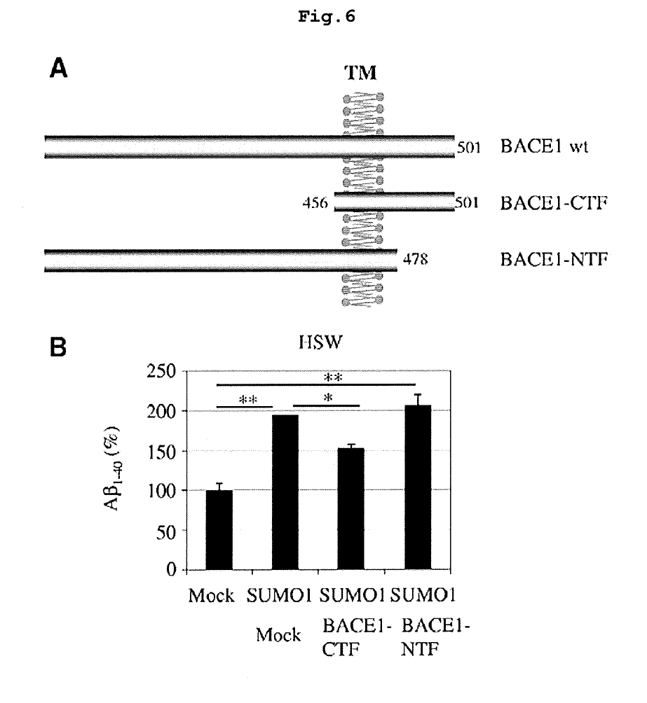

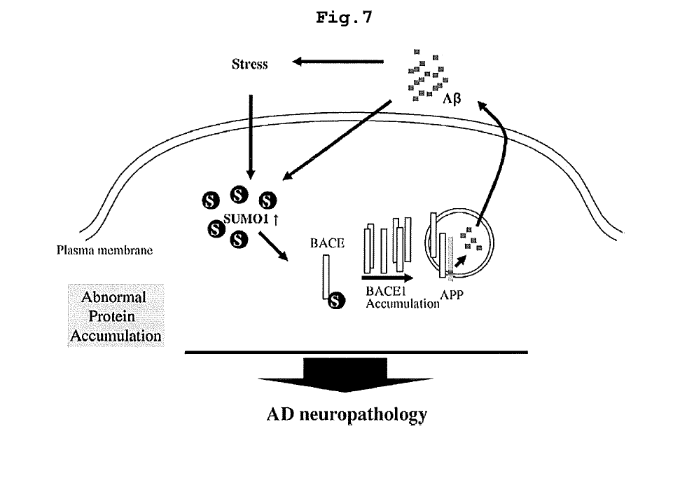

The present inventors further investigated whether the SUMO1-induced Aβ production was a result of an interaction with BACE1 and SUMO1. As a result, it was confirmed that the Aβ production was reduced in the cells transfected with SUMO1 and N-terminal deleted BACE1 (see FIG. 6).

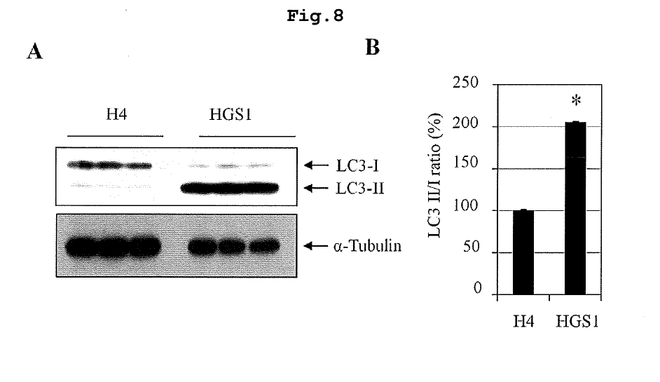

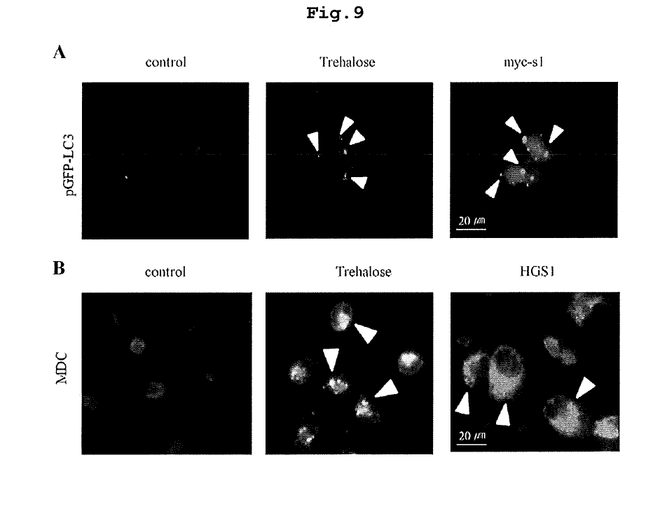

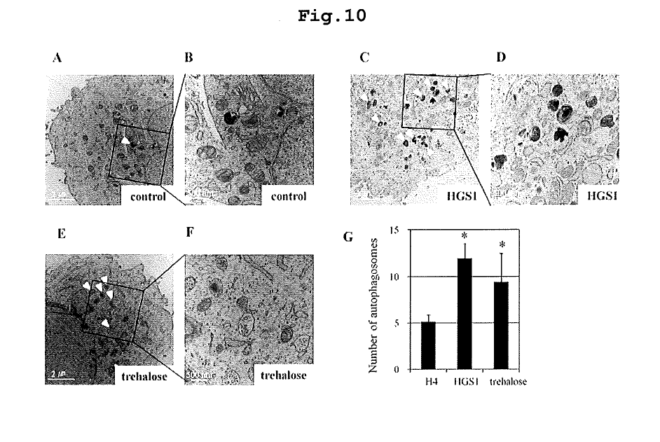

It was additionally confirmed that the levels of LC3-II were increased (see FIG. 8) and the formation of autophagosome was activated (see FIG. 9) in the presence of SUMO1. The autophagosome formation was significantly increased in the cells transfected with SUMO1 (see FIG. 10).

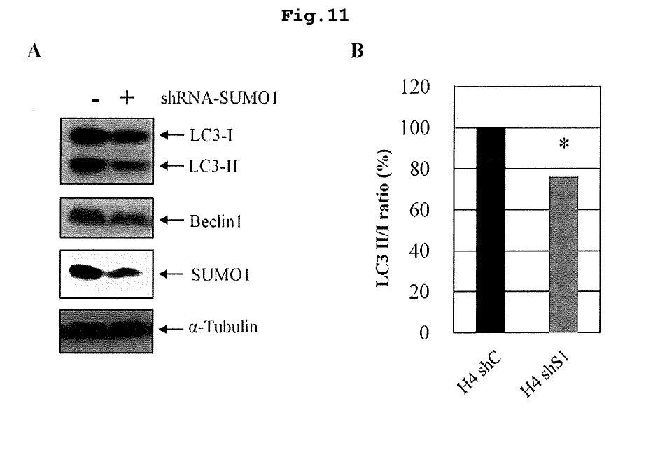

It was also confirmed that the depletion of SUMO1 inhibited autophagy induction by decreasing the levels of LC3-II in H4 cells (see FIG. 11).

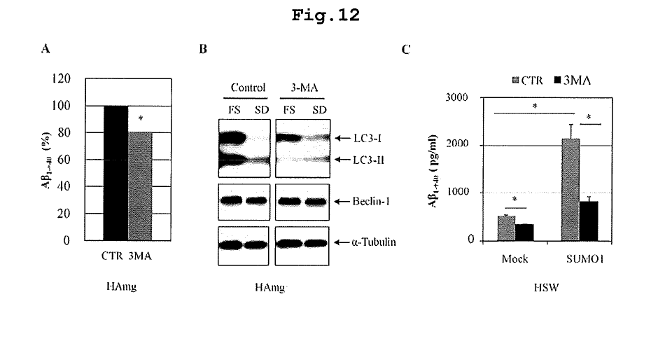

When the cells expressing Swedish mutant-type APP695 were treated with 3-methyladenine inhibiting Aβ generation, autophagosome formation was suppressed, suggesting that SUMO regulated Aβ level via autophagy activation pathway (see FIG. 12).

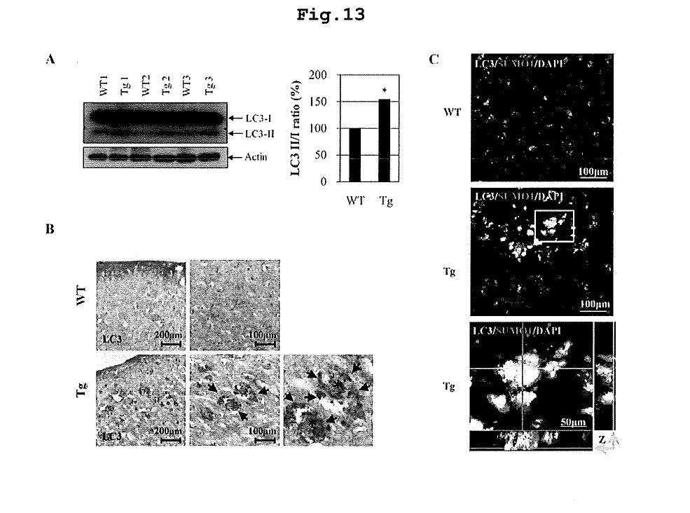

It was also confirmed that the levels of SUMO1 and LC3 were increased and they were co-localized in the APP transgenic mice, suggesting that SUMO was involved in the autophagosome induction pathway (see FIG. 13).

Therefore, the present invention confirmed that SUMO1 increased BACE1 accumulation and Aβ generation and interacted with BACE1 to regulate BACE1 accumulation, and further confirmed that SUMO1 depletion decreased LC3-II expression. The above confirmation indicated that the inhibitor of SUMO1 and BACE1 interaction or the inhibitor of SUMO1 expression could be effectively used for the treatment of degenerative brain disease.

The composition comprising the inhibitor of SUMO1 and BACE1 interaction or the inhibitor of SUMO1 expression or activation of the present invention can include, in addition to the above ingredients, one or more effective ingredients having the same or similar function to the same.

The composition of the present invention can additionally include a pharmaceutically acceptable additive, which is exemplified by starch, gelatinized starch, microcrystalline cellulose, lactose, povidone, colloidal silicon dioxide, calcium hydrogen phosphate, lactose, mannitol, taffy, Arabia rubber, pre-gelatinized starch, corn starch, cellulose powder, hydroxypropyl cellulose, Opadry, sodium carboxy methyl starch, carunauba wax, synthetic aluminum silicate, stearic acid, magnesium stearate, aluminum stearate, calcium stearate, white sugar, dextrose, sorbitol, talc, etc. The pharmaceutically acceptable additive herein is preferably added by 0.1˜90 weight part to the composition.

The composition of the present invention can be administered orally or parenterally and be used in general forms of pharmaceutical formulation. The composition of the present invention can be prepared for oral or parenteral administration by mixing with generally used diluents or excipients such as fillers, extenders, binders, wetting agents, disintegrating agents and surfactant. Solid formulations for oral administration are tablets, pills, powders, granules and capsules. These solid formulations are prepared by mixing the inhibitor of SUMO1 and BACE1 interaction or the inhibitor of SUMO1 expression or activation with one or more suitable excipients such as starch, calcium carbonate, sucrose or lactose, gelatin, etc. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations, suppositories and injections. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The composition of the present invention can be administered orally or parenterally. The parenteral administration is exemplified by skin external application, intraperitoneal injection, intralectal injection, subcutaneous injection, intravenous injection, intramuscular injection and intrathoracic injection. The effective dosage of the composition can be determined according to weight, age, gender, health condition, diet, administration frequency, administration method, excretion and severity of disease.

The dosage unit can contain, for example, 1, 2, 3 or 4 individual doses or ½, ⅓ or ¼ of an individual dose. An individual dose preferably contains the amount of active compound which is administered in one application and which usually corresponds to a whole, ½, ⅓ or ¼ of a daily dose. The effective dosage of the composition of the present invention is preferably 0.0001˜10 g/kg per day, and more preferably 0.001˜1 g/kg per day. The administration frequency is preferably 1˜6 times a day. However, the dosage can be changed according to administration way, severity of disease, gender, weight, age, etc, so the above dosage cannot limit the scope of the invention in any way.

The composition of the present invention can be administered alone or together with surgical operation, hormone therapy, chemo-therapy and biological regulators to prevent and treat neurodegenerative disease.

The present invention provides a diagnostic method for degenerative brain disorders comprising the following steps:

1) measuring the level of SUMO1 and BACE1 interaction in a sample separated from a test subject;

2) selecting a subject demonstrating the increased level of SUMO1 and BACE1 interaction, measured in step 1), compared with that of the normal control; and

3) evaluating the risk of degenerative brain disorders in the selected subject of step 2).

The present invention also provides a diagnostic method for degenerative brain disorders comprising the following steps:

1) measuring the level of SUMO1 expression in a sample separated from a test subject;

2) selecting a subject demonstrating the increased level of SUMO1 expression, measured in step 1), compared with that of the normal control; and

3) evaluating the risk of degenerative brain disorders in the selected subject of step 2).

In the above diagnostic method, the test subject of step 1) is preferably a subject suspected to have degenerative brain disease, but not always limited thereto. The sample obtained from the test subject is preferably serum, plasma, or blood, but not always limited thereto.

In the above diagnostic method, the SUMO1 of step 1) is preferably composed of the amino acid sequence represented by SEQ. ID. NO: 1, but not always limited thereto.

In the above diagnostic method, the full length BACE1 of step 1) is composed of the amino acid sequence represented by SEQ. ID. NO: 2, or the C-terminal deleted sequence from the original BACE1 sequence represented by SEQ. ID. NO: 4, but not always limited thereto.

In the above diagnostic method, the measurement of SUMO1 and BACE1 interaction level in step 2) is preferably performed by the method selected from the group consisting of immunofluorescence method, mass spectrometry, protein chip assay, Western blotting, and ELISA, but not always limited thereto.

In the above diagnostic method, the measurement of SUMO1 expression level in step 2) is preferably performed by the method selected from the group consisting of RT-PCR, DNA chip assay, immunofluorescence method, Western blotting, and ELISA, but not always limited thereto.

In the above diagnostic method, the degenerative brain disorders of step 3) is preferably is preferably the one induced by the accumulation of Aβ (β-amyloid), which is preferably the one selected from the group consisting of dementia, Alzheimer's disease, stroke, Huntington's disease, Pick's disease, and Creutzfeldt-Jakob disease, and more preferably the one selected from the group consisting of dementia, Alzheimer's disease, and Huntington's disease.

The present invention provides a screening method for candidates for treating degenerative brain disorders comprising the following steps:

1) treating a test material to cells expressing SUMO1 and BACE1;

2) measuring the level of SUMO1 and BACE1 interaction in the cells of step 1); and

3) selecting a test material that was able to reduce the level of SUMO1 and BACE1 interaction, measured in step 2), compared with the non-treated control.

The present invention also provides a screening method for candidates for treating degenerative brain disorders comprising the following steps:

1) treating a test material to cells expressing SUMO1 and BACE1;

2) measuring the level of SUMO1 expression in the cells of step 1); and

3) selecting a test material that was able to reduce the level of SUMO1 expression, measured in step 2), compared with the non-treated control.

The present invention also provides a screening method for candidates for treating degenerative brain disorders comprising the following steps:

1) treating a test material to the cells obtained from a test subject;

2) measuring the level of SUMO1 and BACE1 interaction in the cells of step 1); and

3) selecting a test material that was able to reduce the level of SUMO1 and BACE1 interaction, measured in step 2), compared with the non-treated control.

The present invention also provides a screening method for candidates for treating degenerative brain disorders comprising the following steps:

1) treating a test material to the cells obtained from a test subject;

2) measuring the level of SUMO1 expression in the cells of step 1); and

3) selecting a test material that was able to reduce the level of SUMO1 expression, measured in step 2), compared with the non-treated control.

The present invention also provides a screening method for candidates for treating degenerative brain disorders comprising the following steps:

1) treating a test material to SUMO1 and BACE1 proteins;

2) measuring the level of SUMO1 and BACE1 interaction in the cells of step 1); and

3) selecting a test material that was able to reduce the level of SUMO1 and BACE1 interaction, measured in step 2), compared with the non-treated control.

The present invention also provides a screening method for candidates for treating degenerative brain disorders comprising the following steps:

1) treating a test material to SUMO1 and BACE1 proteins;

2) measuring the level of SUMO1 expression in the cells of step 1); and 3) selecting a test material that was able to reduce the level of SUMO1 expression, measured in step 2), compared with the non-treated control.

In the above screening method, the SUMO1 of step 1) is preferably composed of the amino acid sequence represented by SEQ. ID. NO: 1, but not always limited thereto.

In the above screening method, the full length BACE1 of step 1) is composed of the amino acid sequence represented by SEQ. ID. NO: 2, or the C-terminal deleted sequence from the original BACE1 sequence represented by SEQ. ID. NO: 4, but not always limited thereto.

In the above screening method, the measurement of SUMO1 and BACE1 interaction level in step 2) is preferably performed by the method selected from the group consisting of immunofluorescence method, mass spectrometry, protein chip assay, Western blotting, and ELISA, but not always limited thereto.

In the above screening method, the measurement of SUMO1 expression level in step 2) is preferably performed by the method selected from the group consisting of RT-PCR, DNA chip assay, immunofluorescence method, Western blotting, and ELISA, but not always limited thereto.

In the above screening method, the degenerative brain disorders of step 3) is preferably is preferably the one induced by the accumulation of Aβ (β-amyloid), which is preferably the one selected from the group consisting of dementia, Alzheimer's disease, stroke, Huntington's disease, Pick's disease, and Creutzfeldt-Jakob disease, and more preferably the one selected from the group consisting of dementia, Alzheimer's disease, and Huntington's disease.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### SUMO1 Levels in APP Transgenic Mice

### <1-1> Preparation of Test Animal

Eighteen-month-old APP transgenic mice were used in the present invention, as previously reported (Kim et al., 2010). Eight-month-old 5×FAD transgenic (Tg) mice [B6SJL Tg (APPSwF1Lon, PSEN1*M146L*L286V) 6799Vas/J] were purchased from the Jackson Laboratory and maintained by crossing hemizygous transgenic mice with B6SJL F1 mice. All studies were conducted with a protocol approved by the local Institutional Animal Care Use Committee in compliance with Korean Food and Drug Administration guidelines for the care and use of experimental animals.

### <1-2> SUMO1 Up-Regulation in APP Transgenic Mice

To determine the relationship between amyloid plaques which were found in the brains of AD patients and SUMO levels, Western blot analysis was performed using α-SUMO1 (Zymed, San Francisco, Calif., USA or Cell Signaling Technology, Danvers, Mass.), α-SUMO2/3 (Zymed), and α-tubulin (Sigma).

For tissue sampling, 18-month-old APP Swedish/PS1Δ9 Tg mice were sacrificed, and the temporal cortex and cerebellum were extracted. At this time, 18-month-old wild type mice were used as the control. Tissue samples were chopped into small pieces and sequentially processed by sonication in 1×RIPA buffer. The protein concentrations were determined using the bicinchoninic acid (BCA) method. Then, equal amount of proteins were separated on NuPAGE (4˜12%; Invitrogen) gels and were transferred to nitrocellulose membranes (Amersham Biosciences, Buckinghamshire, UK). Membranes were blocked in 5% fat-free milk in Tris-buffered saline (TBS) with 0.1% Tween 20 and incubated with the primary antibody overnight at 4° C. Membranes were developed using the enhanced chemiluminescence (ECL) method (Pierce, Rockford, Ill., USA).

As a result, as shown in FIG. 1A and FIG. 1B, it was confirmed that SUMO1 protein levels were specifically increased in the APP Swedish/PS1ΔE9 Tg mouse brain cells (FIG. 1A). Quantification of the SUMO1 band demonstrated that SUMO1 levels in the APP Swedish/PS1ΔE9 Tg mouse brain cells were increased up to 200% compared with those in the control group (*p<0.05; FIG. 1B).

### <1-3> Localization of Amyloid Plaques and SUMO Expression in APP Transgenic Mice

To determine the relationship between amyloid plaques and SUMO expression sites, immuno-staining was performed.

Particularly, brains from 18-month-old APP Swedish/PS1ΔE9 Tg mice together with their wild type controls were fixed in 4% paraformaldehyde. Cryostat sagittal sections were cut on a sliding microtome into 10 um slices at −20° C. and placed on a microslide for immunostaining. The sections were immunostained with 6E10 (1:100; Covance), the monoclonal antibody raised against 1-17 amino acids of Aβ region, and SUMO1 antibodies (1:100; Cell Signaling Technology). Sections were then incubated for 1 hour at room temperature with secondary antibodies conjugated with Alexa Fluor 488 and 555. Axiolab-Pol polarizing (Carl Zeiss, Thornwood, N.Y., USA) microscopy with Axio Vision Release 4.8 software was used for analysis of 3,3′-Diaminobenzidine (DAB) photomicrographs and co-localization of immunofluorescent proteins.

As a result, DAB-stained images of the cortex, hippocampus, and amygdala are shown in FIG. 1C. It was confirmed that SUMO1 immunoreactivity was enhanced in Tg mice. Additionally, SUMO1 and amyloid plaque were double stained to clarify their co-localization. These results suggest that SUMO1 levels are elevated in the AD Tg mouse brain and that some of them aggregate with amyloid plaques (FIG. 1C).

### Example 2

### Up-Regulations of BACE1 and SUMO1 by Aβ₁₋₄₀ Peptides

### <2-1> Cell Lines and Cell Culture

Human neuroglioma H4 cells, mouse neuroblastoma N2a cells or HEK293T cells were cultured in Dulbecco's Modified Eagle's Medium (DMEM) supplemented with 10% fetal bovine serum (FBS) and 1% penicillin/streptomycin. H4 cells stably expressing Swedish familial Alzheimer's disease (FAD) mutant APP695 have been reported previously (Chae et al., 2010). N2a cells stably expressing Swedish FAD mutant APP695 was named NSW. H4 cells stably expressing human wild type BACE1 with Myc tag (HBmg), wild type BACE1 with V5 tag (HBvg) or dileucine (LL/AA) mutant BACE1 with V5 tag (HBLA) were established by G418 selection. HEK293T cells stably expressing Swedish FAD mutant APP695 were named 293SW. For primary cortical neuronal cultures, dissociated cells from cortexes of embryonic rat brains were plated in 12-well dishes coated with poly-D-lysine (0.1 mg/mL) and maintained in neurobasal medium supplemented with B27 (Promega).

### <2-2> Up-Regulation of SUMO1 by Aβ

To determine whether Aβ could influence protein levels in vitro, the present inventors examined the effect of Aβ₁₋₄₀ peptides on protein levels of BACE1 and SUMO1 in primary cultured rat cortical neurons and H4 cells stably expressing wild-type human BACE1 (HBmg).

Particularly, cortical neurons were exposed to Aβ₁₋₄₀ peptides (Invitrogen, USA) for 48 hours, and 20 uM dimethyl sulfoxide (DMSO) was used to treat controls. Cells were washed with PBS and lysed with 1×radioimmunoprecipitation assay (RIPA) buffer. Total proteins were separated by NuPAGE 4-12% bis-tris-polyacrylamide gel electrophoresis using MES SDS running buffer. For quantification, blots were scanned, and the signal densities were measured using NIH Image 5.0 software (National Institutes of Health, Bethesda, Md., USA). Western blotting was performed using α-SUMO1 (Zymed, San Francisco, Calif., USA or Cell Signaling Technology, Danvers, Mass.) and α-tubulin (Sigma) by the same manner as described above.

As a result, it was confirmed that monomer forms of SUMO1 showed a 160% increase at 10 uM and 260% at 20 uM Aβ₁₋₄₀ peptides (*p<0.05; FIG. 2A and FIG. 2B).

For detection of SUMO-conjugated protein, HBmg cells were sonicated after resuspension in sumoylation lysis buffer [50 mM Tris-HCl (pH7.5), 40 mM NaCl, 0.4% Nonidet P-40, 0.4% Na-deoxycholate, 1.4% SDS, 8% glycerol] supplemented with protease inhibitors (Adamson and Kenney, 2001). Western blotting was performed by the same manner as described above.

As a result, as shown in FIG. 2C and FIG. 2D, it was confirmed that administration of Aβ₁₋₄₀ increased SUMO1-conjugated protein dose-dependently (FIG. 2C and FIG. 2D).

### Example 3

### Up-Regulation of BACE1 by SUMO1

### <3-1> No Effect of Sumoylation on SUMO-Mediated BACE1 Elevation

To confirm the up-regulation of BACE1 induced by SUMO1, Western blot analysis was performed using α-BACE1 (ProScience, Flint Place Poway, Calif., USA), α-GFP (Molecular Probes, Eugene, Oreg., USA), and α-tubulin (Sigma).

Particularly, primary cultured rat cortical neurons and H4 cells (HBmg) were transfected with SUMO1, SUMO2, and SUMO3, followed by Western blotting by the same manner as described above.

As a result, the levels of BACE1 were significantly increased in both cortical neurons and HBmg cells (FIG. 3A and FIG. 3B). To confirm whether or not the above result was attributed to sumoylation that changes protein functions via interaction with target proteins, HBmg cells were transfected with conjugation deficient SUMO mutant, followed by Western blotting by the same manner as described above.

As a result, the levels of BACE1 were increased by the expression of conjugation deficient SUMO mutant (FIG. 3C), suggesting that the increase of BACE1 expression was not by sumoylation.

### <3-2> SUMO1 and BACE1 Complex Formation

The present inventors examined whether SUMO interacts with BACE1.

Particularly, H4 cells stably expressing V5-tagged wild type BACE1 (HBvg) were transfected with Myc-tagged SUMO1. Cells were washed with phosphate-buffered saline (PBS) and lysed in immunoprecipitation buffer [50 mM Tris-HCl (pH 8.0), 150 mM NaCl, 2 mM EDTA (pH 8.0), 10% glycerol, 1% Triton X-100] supplemented with protease inhibitor (Sigma). The lysates from each sample set were incubated with protein A/G agarose for 2 hours at 4° C. for preclearing. The precleared lysates were incubated with anti-V5 antibody overnight at 4° C. After the incubation with A/G agarose for 2 hours, the resin was washed, and the pellets were resuspended in sodium dodecyl sulfate (SDS) sample buffer. Then, Western blotting was performed using α-Myc (Cell Signaling Technology) by the same manner as described above.

As a result, as shown in FIG. 3D, it was confirmed that SUMO1 was specifically detected in BACE1-V5 immune complexes (FIG. 3D). These results demonstrate that SUMO1 forms complex with BACE1.

### Example 4

### BACE1 Region Required for the Interaction with SUMO1

To confirm the region of BACE1 required for the interaction with SUMO1, the present inventors investigated whether BACE1 accumulation by SUMO1 was because of the BACE1 dileucine motif known to be involved in protein-protein interactions (Cole and Bassar, 2007).

Particularly, Western blotting and immunoprecipitation were performed using α-V5 (Invitrogen), α-Myc (Cell Signaling Technology) and α-tubulin (Sigma) by the same manner as described above.

When the H4 cells stably expressing the BACE1 dileucine mutant (HBLA) were transfected with Myc-tagged SUMO, BACE1 dileucine mutant (BACE1 mut) protein levels were not increased by over-expression of SUMO (FIG. 3F).

To identify the dileucine motif within BACE1 required for the interaction with BACE1 and SUMO1, HBLA cells expressing the BACE1 dileucine mutant were transfected with Myc-tagged SUMO1. When the lysates were subjected to immunoprecipitation with anti-Myc antibody for SUMO1, wild-type BACE1 (BACE1 wt) coimmunoprecipitated with SUMO1, whereas the BACE1mut failed to coimmunoprecipitate with SUMO1 (FIG. 3E).

These results imply that BACE1 accumulation requires SUMO1, which might interact with the dileucine motif of BACE1.

### Example 5

### Down-Regulation of BACE1 by Depletion of SUMO1

### <5-1> Construction of Small Interfering RNA and Short Hairpin RNA

To determine whether depletion of SUMO1 played a role in wild-type BACE1 accumulation, the present inventors used small interfering RNA and short hairpin RNA.

Particularly, the target sequence of siSUMO1 #1 has been already reported (Dorval et al., 2007) and #2 is 5′-AACACATCTCAAGAAACTC-3′(SEQ. ID. NO: 5). These 2 SUMO1 siRNAs were synthesized as a duplex by Bioneer (Daejeon, Korea). The siRNA against SUMO2 and SUMO3 was purchased from Dharmacon RNA Technology (Lafayette, Colo., USA). shSUMO1, whose target sequence is siSUMO1 #1, was synthesized. Annealing and ligation into pRNA-U6.1/zeo (Genscript, Piscataway, N.J.) was performed according to the supplier's instructions. To confirm the protein level, Western blotting was performed by the same manner as described above.

### <5-2> Down-Regulation of BACE1 by Depletion of SUMO1

SUMO1 was knocked down using the shRNA constructed above, and then BACE1 levels were investigated in HBmg and HBLA cells. At this time, shRNA vector was used as the negative control. 60 hours after the transfection, Western blotting was performed using α-Myc (Cell Signaling Technology) and α-V5 (Invitrogen).

As a result, as shown in FIG. 4A and FIG. 4B, approximately 30% decrease in wild-type BACE1 protein levels was observed in HBmg cells (FIG. 4A). However, BACE1 dileucine mutant protein was not down-regulated in HBLA cells (FIG. 4B). These results suggest that BACE1 down-regulation by SUMO1 depletion is also dependent on the dileucine motif of BACE1.

Next, the present inventors investigated whether SUMO1 depletion could inhibit wild-type BACE1 up-regulation by Aβ or staurosporine (STS).

After HBmg cells were transfected with SUMO1 shRNA for 24 hours, cells were treated with 10 uM Aβ₁₋₄₀. Then, Western blot analysis was performed using α-Myc (Cell Signaling Technology), α-SUMO1 (Zymed, San Francisco, Calif., USA or Cell Signaling Technology, Danvers, Mass.), and α-GGA3 (BD Transduction Laboratories, San Jose, Calif., USA).

As a result, as shown in FIG. 4C, it was confirmed that Aβ₁₋₄₀-induced BACE1 elevation was inhibited by transfection of SUMO1 shRNA (FIG. 4C). This result suggests that the important regulator corresponding to Aβ₁₋₄₀-induced BACE1 elevation might be SUMO1.

The present inventors also examined the role of SUMO1 in STS-mediated BACE1 accumulation. Particularly, HBmg cells were treated with 0.1, 1, or 2 uM STS for 18 hours and then SUMO1 protein levels were investigated. HBmg cells were also treated with shSUMO1 for 48 hours, and then treated with 1 uM STS for 18 hours to confirm STS-induced BACE1 accumulation. Western blot analysis was performed using α-SUMO1 (Zymed, San Francisco, Calif., USA or Cell Signaling Technology, Danvers, Mass.), α-Myc (Cell Signaling Technology), and α-actin (Sigma). As a result, protein levels of SUMO1 were increased during apoptosis (FIG. 4D), and STS-induced BACE1 accumulation was inhibited by SUMO1 shRNA (FIG. 4E).

The present inventors also investigated whether STS-induced apoptosis could induce BACE1 accumulation in SUMO1^{-/-} MEF obtained from SUMO1 knockout mice (Dr. Olli A. Janne, University of Helsinki, Finland).

Particularly, SUMO1^{-/-} MEF was treated with 1 uM STS for 18 hours. Then, Western blot analysis was performed using α-BACE1 (ProScience, Flint Place Poway, Calif., USA) and α-tubulin (Sigma). For quantification, NIH Image 5.0 software (National Institutes of Health, Bethesda, Md., USA) was used.

As a result, as shown in FIG. 4F, increased BACE1 protein levels by STS-induced apoptosis were observed in SUMO1^{+/+} MEF, but not SUMO1^{-/-} MEF (FIG. 4F). Therefore, it was confirmed that wild-type BACE1 protein levels were decreased by SUMO1 depletion.

### Example 6

### Increase of Aβ Generation by SUMO1

To investigate the role of the 3 SUMO isoforms in amyloid precursor protein (APP) processing, the present inventors tested whether SUMO1, SUMO2 or SUMO3 induced an increase in Aβ.

Particularly, HBmg cells were transiently co-transfected with SUMO1, SUMO2 or SUM03 and wild type APP. Then, secreted Aβ₁₋₄₀ was measured in conditioned medium using commercial human Aβ enzyme-linked immunosorbent assay (ELISA) kits (BioSource International, Camarillo, Calif., USA) according to the manufacturer's instructions.

As a result, as shown in FIG. 5A and FIG. 5B, Aβ₁₋₄ levels were increased when SUMO1, SUMO2 or SUMO3 was co-expressed with wild-type APP in cells (FIG. 5A). However, this result was not observed in HBLA cells expressing dileucine mutant BACE1 (FIG. 5B). Therefore, it was confirmed that Aβ elevation by SUMO1, SUMO2 or SUMO3 over-expression is dependent on BACE1 regulation. These results suggest that SUMO might regulate Aβ levels via the dileucine motif of BACE1.

### Example 7

### Down-Regulation of Aβ₁₋₄₀ by Depletion of SUMO1

To investigate whether depletion of SUMO1 could decrease Aβ₁₋₄₀ levels, ELISA was performed using shRNA.

Particularly, H4 cells co-expressing wild-type APP and wild-type BACE1 or dileucine mutant BACE1 were transfected with or without shRNA of SUMO1. 48 hours later, ELISA was performed according to same manner.

As a result, as shown in FIG. 5C and FIG. 5D, SUMO1 depletion decreased Aβ₁₋₄₀ levels in H4 cells expressing wild-type APP and wild-type BACE1 (FIG. 5C). However, SUMO1 depletion did not change Aβ₁₋₄₀ levels in H4 cells expressing wild-type APP and dileucine mutant BACE1 (FIG. 5D). These results suggest that the alteration of Aβ by SUMO1 depletion is due to change in not APP proteins but BACE1.

### Example 8

### Down-Regulation of Aβ by N-Terminal Deleted BACE1 (BACE1-CTF)

To investigate whether the SUMO1-induced Aβ production was a result of an interaction with BACE1 and SUMO1, the present inventors engineered deletion mutants of BACE1 [N-terminal deleted BACE1-CTF (amino acid sequence 456-501, SEQ. ID. NO: 3) and C-terminal deleted BACE1-NTF (amino acid sequence 1-478, SEQ. ID. NO: 4)] (FIG. 6A).

Particularly, to construct the BACE1-CTF, pEGFP-N3 (Clontech, USA) was digested with ApaI, and dephosphorylated using CiP (Calf instestinal alkaline phosphatases) to obtain a backbone. DNA was separated by electrophoresis, followed by extraction using gel extraction kit (Qiagen, USA). PCR was performed using pcDNA3-VACE1-v5-his as a template with the forward primer containing ApaI restriction enzyme site (BACE1-CTF_F, SEQ. ID. NO: 6) and the reverse primer (BACE1-CTF_R, SEQ. ID. NO: 7). The PCR product was digested with ApaI, followed by extraction using DNA electrophoresis and gel extraction. The backbone vector and the PCR product were ligated using T4 DNA ligase, followed by DNA sequencing.

To construct the BACE1-NTF, pcDNA3.1-v5-his was digested with Xhol and BamHI to obtain a backbone vector. DNA was separated by electrophoresis, followed by extraction using gel extraction kit (Qiagen, USA). PCR was performed using pcDNA3-VACE1-v5-his as a template with the forward primer (BACE1-NTF_F, SEQ. ID. NO: 8) and the reverse primer containing XhoI restriction enzyme site (BACE1-NTF_R, SEQ. ID. NO: 9). The PCR product was digested with BamHI/XhoI, followed by extraction using DNA electrophoresis and gel extraction. The backbone vector and the PCR product were ligated using T4 DNA ligase, followed by DNA sequencing.

ELISA was performed to measure the Aβ production induced by the constructed mutants BACE1-NTF and BACE1-CTF.

Particularly, HSW cells were transfected with combinations of SUMO1, BACE1-CTF and BACE1-NTF constructs. Then, the secreted Aβ levels were measured by ELISA. At this time HSW cells transfected with SUMO1 alone were used as the control.

As a result, as shown in FIG. 6B, Aβ production in cells transfected with SUMO1/BACE1-CTF was reduced by approximately 25%, compared with the control. However, Aβ production in cells transfected with SUMO1/BACE1-NTF was not changed compared with the control (FIG. 6B). Therefore, it was confirmed that the above peptides inhibited SUMO1 and BACE1 interaction and hence significantly reduced Aβ production. Thus, the peptides were confirmed to be effectively used as a composition for the prevention and treatment of degenerative brain disease.

### Example 9

### Autophagy Induction in H4 Cells by SUMO Proteins

### <9-1> LC3-II Up-Regulation by SUMO1

To investigate the role of SUMO1 protein in inducing autophagy, the present inventors quantified the level of LC3-II protein in H4 cells and H4 cells stably expressing SUMO1 (HGS1 cells) by using Western blotting.

Particularly, Western blotting was performed by the same manner as described in Example <2-2>. At this time, the following primary antibodies were used: LC3 (MBL, USA) and α-Tubulin (Sigma, USA).

As a result, as shown in FIG. 8, the levels of LC3-II were significantly increased in HGS1 cells (FIG. 8A), and the LC3-II/I ratio was increased up to 210% (p<0.05; FIG. 8B).

### <9-2> Activation of Autophagosome Formation by SUMO1

To verify whether autophagosome formation is activated by SUMO1, cells were stained with monodansylcadaverine (MDC), a fluorescent dye that is incorporated selectively into autophagosomes and autolysosomes.

Particularly, H4 cells were transiently transfected with GFP-tagged LC3 or transiently co-transfected with GFP-tagged LC3 and myc-tagged SUMO1, and then incubated with or without 100 mM trehalose for 24 hours. Cells were washed twice with PBS and incubated with 50 μM MDC in PBS at 37° C. for 30 minutes. After incubation, the cells were washed three times with PBS and analyzed immediately using an fluorescent microscope (Olympus, Japan) (excitation wavelengths 380-420, barrier filter 450 nm).

As a result, as shown in FIG. 9A, autophagic markers were dispersed as small dots throughout the cytosol of H4 cells. When the H4 cells were treated with trehalose, more LC3-II-positive dots were observed (FIG. 9A). When the cells were transiently co-transfected with LC3 and SUMO1, LC3-II-positive autophagosomes were increased as much as in the trehalose-treated positive control. In particular, when trehalose was treated, enlarged MDC-positive autophagosome were significantly increased with much stronger intensity throughout the entire cell body (FIG. 9B).

### <9-3> Increase of Autophagosome Formation by SUMO1

To investigate whether autophagosome formation is increased in cells transfected with SUMO1, electron microscopy was performed.

Particularly, H4 cells were treated with 100 mM of trehalose for 24 hours for the positive control. Cells were fixed overnight in a mixture of cold 2.5% glutaraldehyde (EMS, USA) in 0.1 M PBS (pH 7.2) and 2% paraformaldehyde (Merck, USA) in 0.1 M PBS (pH 7.2) and embedded with epoxy resin (EMS, USA). The epoxy resin-mixed samples were loaded into capsules and polymerized at 60° C. Thin sections were sliced on an ultramicrotome (Leica, USA) and collected on a copper grid. Appropriate areas for thin sectioning were cut at 70 nm and stained with saturated 2% uranyl acetate (EMS, USA) before examination on a transmission electron microscope (Carl Zeiss, Germany) at 120 kV.

As a result, as shown in FIG. 10, there were abundant autophagosomes in SUMO1-transfected H4 cells (HGS1) (FIGS. 10C 10D, and 10E). Autophagosome formation was also increased in H4 cells treated with trehalose, the positive control (FIGS. 10E, 10F, and 10G).

Collectively, these results indicate that SUMO1 activates autophagosome formation in H4 cells.

### Example 10

### Down-Regulation of LC3-II in H4 Cells by SUMO1 Depletion

To investigate whether SUMO1 depletion inhibits autophagy induction in H4 cells, cells were transfected with the shRNA used in Example <5-1>, followed by Western blotting by the same manner as described in Example <2-2>. At this time, the following primary antibodies were used: SUMO1 (Cell Signaling, USA), LC3 (MBL, USA), Beclin 1 (Cell Signaling, USA) and α-Tubulin (Sigma, USA).

As a result, as shown in FIG. 11, SUMO1 expression in cells transfected with SUMO1 shRNA was reduced by approximately 60% compared with the control (FIG. 11A). SUMO1 depletion reduced the LC3-II/I ratio in H4 cells by approximately 80% (FIG. 11B). The beclin-1/α-tubulin ratio did not change significantly in H4 cells (data not shown). These results suggest that SUMO1 regulates the autophagy induction pathway

### Example 11

### Increase of Aβ Production in H4 Cells Stably Expressing Swedish Mutant-Type APP695 (HSW) by SUMO

<11-1> Inhibition of Autophagosome Formation by 3-methyladenine (3-MA)

To verify whether autophagy inhibition reduces Aβ secretion, H4 cells were treated with 3-methyladenine known to suppress macroautophagy-induced Aβ generation, followed by Western blotting.

Particularly, for the treatment of 3-methyladenine, cells were incubated in full-serum or serum-depleted medium for 18 hours. After serum depletion, the cells were treated with 3-methyladenine for 24 hours. Western blotting was performed by the same manner as described in Example <2-2> using the primary antibodies against LC-3, Beclin 1, and α-tubulin.

As a result, as shown in FIG. 12B, the LC3-II/I ratio was significantly higher in cells after incubation of serum-depletion and this change was blocked by the treatment of 3-methyladenine (FIG. 12B).

### <11-2> SUMO1-Induced Aβ Production by Autophagic Activation

To investigate whether SUMO1-induced Aβ production was due to autophagic activation, ELISA was performed.

Particularly, HA cells or HSW cells transfected with SUMO1 were treated 3-methyladenine for 24 hours. Then, secreted Aβ1-40 was measured in culture medium using SoftMax Pro 5.0 software. Concentrations of Aβ1-40 was quantified using commercial human Aβ ELISA kits (Invitrogen, USA) according to the manufacturer's instructions.

As a result, as shown in FIG. 12A, the level of secreted Aβ1-40 was reduced by about 80% compared to the control by the treatment of 3-methyladenine (FIG. 12A). In the meantime, the production of Aβ1-40 in HSW cells transfected with SUMO1 increased by about 450% compared to the control (FIG. 12C).

These results suggest that SUMO1 regulates Aβ levels via the autophagy induction pathway in HSW cells.

### Example 12

### Expression Sites of SUMO1 and LC3 in APP Transgenic Mice

### <12-1> LC3-II Elevation in APP Transgenic Mice

To investigate LC3-II expression levels in APP transgenic mice (AD model mice), Western blotting was performed by the same manner as described in Example <2-2> using the primary antibodies against LC3 and Actin.

As a result, as shown in FIG. 13A, the LC3-II/I ratio was significantly increased in APP transgenic mice. This result indicates that LC3-II is accumulated in the brain of the mice.

<12-2> Expression sites of SUMO1 and LC3 in APP Transgenic Mice

To investigate the relationship between SUMO1 and LC3 elevation in APP transgenic mice, immunostaining was performed.

Particularly, immunostaining was performed by the same manner as described in Example <1-3> using the primary antibodies against LC-3 (1:200, Abgent, USA) and SUMO1 (1:100, Cell Signaling, USA).

As a result, as shown in FIG. 13B, LC3 expression was increased in APP transgenic mice. It was also confirmed that some of amyloid plaques stained by Congo Red were surrounded by LC3 positive cells (FIG. 13B). When the APP transgenic mouse cortex was investigated by double-label immunofluorescence confocal microscopy, immunoreactivity of LC3 was co-localized with that of SUMO1, supporting that SUMO1 may be involved in the autophagosome induction pathway.

### Manufacturing Example 1

### Preparation of Pharmaceutical Formulations

### <1-1> Preparation of Powders

| | |
|---|---|
| The inhibitor of SUMO1 and BACE1 | 2 g |
| interaction of the present invention | |
| Lactose | 1 g |

Powders were prepared by mixing all the above components, which were filled in airtight packs according to the conventional method for preparing powders.

### <1-2> Preparation of Tablets

| | |
|---|---|
| The inhibitor of SUMO1 and BACE1 | 100 mg |
| interaction of the present invention | |
| Corn starch | 100 mg |
| Lactose | 100 mg |
| Magnesium stearate | 2 mg |

Tablets were prepared by mixing all the above components by the conventional method for preparing tablets.

### <1-3> Preparation of Capsules

| | |
|---|---|
| The inhibitor of SUMO1 and BACE1 | 100 mg |
| interaction of the present invention | |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

### <1-4> Preparation of Pills

| | |
|---|---|
| The inhibitor of SUMO1 and BACE1 | 1 g |
| interaction of the present invention | |
| Lactose | 1.5 g |
| Glycerin | 1 g |
| Xylitol | 0.5 g |

Pills were prepared by mixing all the above components according to the conventional method for preparing pills. Each pill contained 4 g of the mixture.

### <1-5> Preparation of Granules

| | |
|---|---|
| The inhibitor of SUMO1 and BACE1 | 150 mg |
| interaction of the present invention | |
| Soybean extract | 50 mg |
| Glucose | 200 mg |
| Starch | 600 mg |

All the above components were mixed, to which 100 mg of 30% ethanol was added. The mixture was dried at 60° C. and the prepared granules were filled in packs.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A treatment method for degenerative brain disorders containing the step of administering a pharmaceutically effective dose of the inhibitor of SUMO1 (small ubiquitin-like modifier 1) and BACE1 (β-secretase) interaction or the inhibitor of SUMO1 expression or activation to a subject having degenerative brain disorders.

2. The treatment method for degenerative brain disorders according to claim 1, wherein the SUMO1 is composed of the amino acid sequence represented by SEQ. ID. NO: 1.

3. The treatment method for degenerative brain disorders according to claim 1, wherein the full length BACE1 is composed of the amino acid sequence represented by SEQ. ID. NO: 2, or the C-terminal deleted sequence from the original BACE1 sequence represented by SEQ. ID. NO: 4.

4. The treatment method for degenerative brain disorders according to claim 1, wherein the inhibitor of SUMO1 and BACE interaction is selected from the group consisting of peptides binding complementarily to SUMO1 or BACE1, peptides binding complementarily to the fragment having C-terminal deleted from the BACE1 sequence represented by SEQ. ID. NO: 3, peptide mimetics, substrate analogs, aptamers and antibodies.

5. The treatment method for degenerative brain disorders according to claim 1, wherein the inhibitor of SUMO1 expression or activation is selected from the group consisting of antisense nucleotide binding complementarily to SUMO1 mRNA, siRNA, and shRNA.

6. The treatment method for degenerative brain disorders according to claim 1, wherein the inhibitor of SUMO1 expression or activation is selected from the group consisting of peptides binding complementarily to SUMO1 protein, peptide mimetics, substrate analogs, aptamers and antibodies.

7. The treatment method for degenerative brain disorders according to claim 1, wherein the degenerative brain disorders is selected from the group consisting of dementia, Alzheimer's, stroke, Huntington's disease, Pick's disease, and Creutzfeldt-Jakob disease.

8. A diagnostic method for degenerative brain disorders comprising the following steps:
1) measuring the level of SUMO1 and BACE1 interaction or the level of SUMO1 expression in a sample separated from a test subject;
2) selecting a subject demonstrating the increased level of SUMO1 and BACE1 interaction or the increased level of SUMO1 expression, measured in step 1), compared with that of the normal control; and
3) evaluating the risk of degenerative brain disorders in the selected subject of step 2).

9. The diagnostic method for degenerative brain disorders according to claim 8, wherein the sample is selected from the group consisting of serum, plasma, and blood.

10. The diagnostic method for degenerative brain disorders according to claim 8, wherein the measurement of the level of SUMO1 and BACE1 interaction is performed by the method selected from the group consisting of immunofluorescence method, mass spectrometry, protein chip assay, Western blotting, and ELISA.

11. The diagnostic method for degenerative brain disorders according to claim 8, wherein the measurement of the level of SUMO1 expression is performed by the method selected from the group consisting of RT-PCR, DNA chip assay, immunofluorescence method, Western blotting, and ELISA.

12. The diagnostic method for degenerative brain disorders according to claim 8, wherein the degenerative brain disorders is selected from the group consisting of dementia, Alzheimer's, stroke, Huntington's disease, Pick's disease, and Creutzfeldt-Jakob disease.

13. A screening method for candidates for treating degenerative brain disorders comprising the following steps:
1) treating a test material to the cells expressing SUMO1 and BACE1;
2) measuring the level of SUMO1 and BACE1 interaction or the level of SUMO1 expression in the cells of step 1); and
3) selecting a test material that was able to reduce the level of SUMO1 and BACE1 interaction or the level of SUMO1 expression, measured in step 2), compared with the non-treated control.

14. The screening method for candidates for treating degenerative brain disorders according to claim 13, wherein the measurement of the level of SUMO1 and BACE1 interaction is performed by the method selected from the group consisting of immunofluorescence method, mass spectrometry, protein chip assay, Western blotting, and ELISA.

15. The screening method for candidates for treating degenerative brain disorders according to claim 13, wherein the measurement of the level of SUMO1 expression is performed by the method selected from the group consisting of RT-PCR, DNA chip assay, immunofluorescence method, Western blotting, and ELISA.

16. The screening method for candidates for treating degenerative brain disorders according to claim 13, wherein the degenerative brain disorders is selected from the group consisting of dementia, Alzheimer's, stroke, Huntington's disease, Pick's disease, and Creutzfeldt-Jakob disease.
